SEQUENCE LISTING (6 sequences)

SEQ ID NO: 1 - DNA, 717 bp, Saccharomyces sp.
atggaatacgagaagtttctattgttcggggattccatcactgaatttgcattcaatactaggcccacagaggatgataaagattattatgctcttggagccgcgttggttaacgagtatacaagaaaaatggacatccttcagcgaggattcaaagggtacaactccaagtgggcactgaaggttcttcctgaaatcttaaacaatgagcccaacattgccatggccaccatctttttcggtgccaacgatgcatgcttagctggccctcaatgtgtgcctctcttagaatttgtcgataatataggtcaaatggtgtctgtcatgaaagctcatcacgttcttcctattattgtaggcccggcactggtggatagagagaagtgggaaaaagcaagggctgacgaaacagctctcggatatgtgcgtaccaacgagaactttacagtctattctgatgcgttagcaaagctggccgacgacgaaaaaatccccttcgtgaacttgaataaagcgtttcaaaagatagacggtgatgcctggaagagcctactaacggatggactacacttctctggggaaggatacaaaatcttccatgatgaattgatcaaagccatcgaggcacactatcctcaataccatcctaataacatggaatacaaactaacagactggagagatgtgttggatgacggctccaacattttgtcttaa

SEQ ID NO: 2 - protein, 238 aa, Saccharomyces sp.
MEYEKFLLFGDSITEFAFNTRPTEDDKDYYALGAALVNEYTRKMDILQRGFKGYNSKWALKVLPEILNNEPNIAMATIFFGANDACLAGPQCVPLLEFVDNIGQMVSVMKAHHVLPIIVGPALVDREKWEKARADETALGYVRTNENFTVYSDALAKLADDEKIPFVNLNKAFQKIDGDAWKSLLTDGLHFSGEGYKIFHDELIKAIEAHYPQYHPNNMEYKLTDWRDVLDDGSNILS

SEQ ID NO: 3 - DNA, 40 bp, Artificial Sequence
  Primer
gagctcatagcggccatggaatacgagaagtttctattgt

SEQ ID NO: 4 - DNA, 42 bp, Artificial Sequence
  Primer
ggatcctatgcggccgcctcattattgtttttttccctgaaa

SEQ ID NO: 5 - DNA, 70 bp, Artificial Sequence
  Primer
aggtaccttgggcattatcaattttttggcgcattacattgtagtggaacccttgacagtcttgacgtgc

SEQ ID NO: 6 - DNA, 70 bp, Artificial Sequence
  Primer
gggcgttatgtgaagggaaaataagtcaatacgtacaaaaggactactgccgcacttaacttcgcatctg

(19) United States
(12) Patent Application Publication
(10) Pub. No.: US 2009/0246316 A1
(43) Pub. Date: Oct. 1, 2009

(54) Esterase gene and use thereof

(75) Inventors: Yoshihiro Nakao, Osaka (JP); Yukiko Kodama, Osaka (JP); Tomoko Shimonaga, Osaka (JP)

Correspondence Address: DRINKER BIDDLE & REATH (DC), 1500 K STREET, N.W., SUITE 1100, WASHINGTON, DC, 20005-1209, US

(73) Assignee: Suntory Limited, Osaka (JP)

(21) Appl. No.: 11/991,065

(22) PCT Filed: Sep. 12, 2006
(86) PCT No.: PCT/JP2006/318466

§ 371 Date: Feb. 27, 2008

(30) Foreign Application Priority Data
Sep. 13, 2005 (JP) 2005-265918
Feb. 23, 2006 (JP) 2006-047561

(51) Int. Cl. C12Q 1/68; C12N 9/16; C12N 15/74; C12N 1/19; G01N 33/569; C12C 11/09; C12G 1/073; C12Q 1/44; C12Q 3/00; C12R 1/645

(52) U.S. Cl. 426/15; 536/23.2; 435/196; 435/320.1; 435/254.2; 435/6; 435/7.31; 426/11; 426/16; 426/592

(57) ABSTRACT

The present invention relates to a esterase gene and its uses, specifically, a brewery yeast producing alcoholic beverages with excellent aroma and flavor, alcoholic beverages produced using the yeast, a process for producing the alcoholic beverages. More particularly, the present invention relates to a yeast whose capability of producing ester, which contribute to aroma and flavor of products, was controlled by regulating expression level of IAH1 gene encoding brewery yeast esterase Iah1p, particularly nonScIAH1 gene specific to lager brewing yeast, and to a method for producing alcoholic beverages with the yeast.

### TECHNICAL FIELD

The present invention relates to a esterase gene and to uses of the gene. The invention relates in particular to a brewer's yeast which produces alcoholic beverages with excellent aroma and flavor, alcoholic beverages produced using such a yeast, and a method of producing such alcoholic beverages. More specifically, the invention relates to IAH1 gene which codes for the esterase Iah1p in brewer's yeast, particularly to a yeast whose capability of producing ester, which contribute to aroma and flavor of a product, is controlled by regulating the level of expression of the nonScIAH1 gene characteristic to beer yeast and to a method of producing alcoholic beverages using such a yeast.

### BACKGROUND ART

Esters are an important aromatic component of alcoholic beverages. En the case of rice wine, wine and, whiskey, an increase in the ester content is known to give the beverage a florid aroma as well as cause it to be evaluated highly for its flavor. On the other hand, although esters are an important aromatic component of beer as well, an excess amount of esters is disliked due to the resulting ester smell. Thus, it is important to suitably control the amount of ester, formed according to the type of alcoholic beverage.

Yeast producing high levels of esters have been developed in the past for the purpose of increasing the ester content of alcoholic beverages. Examples of previously reported methods for effective isolation of yeast producing large amounts of esters include a method in which yeast is subjected (or not subjected) to mutagenic treatment to obtain a strain which produces large amounts of caproic acid and is resistant to drugs that inhibit fatty acid synthases such as cerulenin, as well as a strain which is resistant to leucine analogs such as 5,5,5-trifluoro-DL-leucine and produces large amounts of isoamyl alcohol and isoamyl acetate (Japanese Patent Application Laid-open No. 2002-253211), and a method in which a stain is acquired which is grown in medium containing a steroid having a pregnane backbone hydroxylated at position 3 (Japanese Patent Application Laid-open No. 2002-191355).

On the other hand, examples of previously reported methods involving the development of yeast utilizing genetic engineering techniques include expressing high levels of the alcohol acetyl trasferase gene ATF1 of Saccharomyces cerevisiae in brewing yeast (Japanese Patent Application Laid-open No. H06-062849), inhibiting the expression of ATF1 (Japanese Patent Application Laid-open No. H06-253826), and increasing the amount of ester by destroying esterase gene EST2 in brewing yeast (Japanese Patent Application Laid-open No. H09-234077).

### DISCLOSURE OF INVENTION

As stated above, although a mutant strain is acquired for increasing the ester content of a product, there are cases in which unexpected delays in fermentation or increases in undesirable aromatic and flavor components are observed as a result thereof, thus creating problems in the development of yeast for practical application. Consequently, there is need for a method for breeding yeast capable of producing a desired amount of esters without impairing the fermentation rate or product quality.

As a result of conducting extensive studies to solve the above-mentioned problems, the inventors of the present invention succeeded in identifying and isolating a gene from brewer's yeast which encodes an esterase that demonstrates more advantageous effects than known proteins. In addition, the inventors of the present invention also confirmed that the amount of ester formed decreases by producing a transformed yeast by inserting and expressing the resulting gene in yeast, and that the amount of ester formed increases by producing a transformed yeast in which expression of the resulting gene is suppressed, thereby leading to completion of the present invention.

Namely, the present invention relates to a novel esterase gene characteristically present in brewer's yeast, a protein encoded by said gene, a transformed yeast in which the expression of said gene is regulated, and a method for controlling the amount of ester formed in a product by using yeast in which expression of said gene has been regulated. More specifically, the present invention provides the polynucleotide indicated below, a vector containing said polynucleotide, a transformed yeast in which said vector has been inserted, and a method for producing an alcoholic beverage using said transformed yeast.

(1) A polynucleotide selected from the group consisting of:

(a) a polynucleotide comprising a polynucleotide consisting of the nucleotide sequence of SEQ ID NO: 1;

(b) a polynucleotide comprising a polynucleotide encoding a protein consisting of the amino acid sequence of SEQ ID NO:2;

(c) a polynucleotide comprising a polynucleotide encoding a protein consisting of the amino acid sequence of SEQ ID NO:2 with one or more amino acids thereof being deleted, substituted, inserted and/or added, and having a esterase activity,

(d) a polynucleotide comprising a polynucleotide encoding a protein having an amino acid sequence having 60% or higher identity with the amino acid sequence of SEQ ID NO:2, and having a esterase activity,

(e) a polynucleotide comprising a polynucleotide which hybridizes to a polynucleotide consisting of a nucleotide sequence complementary to the nucleotide sequence of SEQ ID NO: 1 under stringent conditions, and which encodes a protein having a esterase activity, and

(f) a polynucleotide comprising a polynucleotide which hybridizes to a polynucleotide consisting of a nucleotide sequence complementary to the nucleotide sequence of the polynucleotide encoding the protein of the amino acid sequence of SEQ ID NO:2 under stringent conditions, and which encodes a protein having a esterase activity.

(2) The polynucleotide of (1) above selected from the group consisting of:

(g) a polynucleotide encoding a protein consisting of the amino acid sequence of SEQ ID NO: 2, or encoding an amino acid sequence of SEQ ID NO: 2 wherein 1 to 10 amino acids thereof is deleted, substituted, inserted, and/or added, and wherein said protein has a esterase activity;

(h) a polynucleotide encoding a protein having 90% or higher identity with the amino acid sequence of SEQ ID NO: 2, and having a esterase activity; and

(i) a polynucleotide which hybridizes to SEQ ID NO: 1 or which hybridizes to a nucleotide sequence complementary to the nucleotide sequence of SEQ. ID NO: 1 under high stringent conditions, and which encodes a protein having a esterase activity

(3) The polynucleotide of (1) above comprising a polynucleotide consisting of SEQ ID NO: 1.

(4) The polynucleotide of (1) above comprising a polynucleotide encoding a protein consisting of SEQ ID NO:2.

(5) The polynucleotide of any one of (1) to (4) above, wherein the polynucleotide is DNA.

(6) A polynucleotide selected from the group consisting of:

(j) a polynucleotide encoding RNA of a nucleotide sequence complementary to a transcript of the polynucleotide (DNA) according to (5) above;

(k) a polynucleotide encoding RNA that represses the expression of the polynucleotide (DNA) according to (5) above through RNAi effect;

(l) a polynucleotide encoding RNA having an activity of specifically cleaving a transcript of the polynucleotide (DNA) according to (5) above; and

(m) a polynucleotide encoding RNA that represses expression of the polynucleotide (DNA) according to (5) above through co-suppression effect.

(7) A protein encoded by the polynucleotide of any one of (1) to (5) above.

(8) A vector comprising the polynucleotide of any one of (1) to (5) above.

(8a) The vector of (8) above, which comprises the expression cassette comprising the following components:

(x) a promoter that can be transcribed in a yeast cell;

(y) any of the polynucleotides described in (1) to (5) above linked to the promoter in a sense direction; and

(z) a signal that can function in a yeast with respect to transcription termination and polyadenylation of a RNA molecule.

(9) A vector comprising the polynucleotide of (6) above.

(10) A yeast, wherein the vector of (8) or (9) above is introduced.

(11) The yeast of (10) above, wherein ester-producing ability is decreased by introducing the vector of (8) above.

(12) A yeast, wherein an expression of the polynucleotide (DNA) of (5) above is repressed by introducing the vector of (9) above, or by disrupting a gene related to the polynucleotide (DNA) of (5) above.

(13) The yeast of (11) above, wherein a ester-producing ability is decreased by increasing an expression level of the protein of (7) above.

(14) A method for producing an alcoholic beverage by using the yeast of any one of (10) to (13) above.

(15) The method for producing an alcoholic beverage of (14) above, wherein the brewed alcoholic beverage is a malt beverage.

(16) The method for producing an alcoholic beverage of (14) above, wherein the brewed alcoholic beverage is a wine.

(17) An alcoholic beverage, which is produced by the method of any one of (14) to (16) above.

(18) A method for assessing a test yeast for its ester-producing capability, comprising using a primer or a probe designed based on a nucleotide sequence of a esterase gene having the nucleotide sequence of SEQ ID NO: 1.

(18a) A method for selecting a yeast having a target ester-producing capability by using the method in (18) above.

(18b) A method for producing an alcoholic beverage (for example, beer) by using the yeast selected with the method in (18a) above.

(19) A method for assessing a test yeast for its ester-producing capability, comprising: culturing a test yeast; and measuring an expression level of a esterase gene having the nucleotide sequence of SEQ ID NO: 1.

(20) A method for selecting a yeast, comprising: culturing test yeasts; quantifying the protein of (7) above or measuring an expression level of a esterase gene having the nucleotide sequence of SEQ ID NO: 1; and selecting a test yeast having said protein amount or said gene expression level according to a target capability of producing ester.

(20a) A method for selecting a yeast, comprising: culturing test yeasts; measuring a ester-producing capability or a esterase activity, and selecting a test yeast having a target capability of producing ester or a target esterase activity.

(21) The method for selecting a yeast of (20) above, comprising: culturing a reference yeast and test yeasts, measuring an expression level of a esterase gene having the nucleotide sequence of SEQ ID NO: 1 in each yeast; and selecting a test yeast having the gene expressed higher or lower than that in the reference yeast.

(22) The method for selecting a yeast of (20) above comprising: culturing a reference yeast and test yeasts; quantifying the protein of (7) above in each yeast; and selecting a test yeast having said protein for a larger or smaller amount than that in the reference yeast. That is, the method for selecting a yeast of (20) above comprising: culturing plural yeasts; quantifying the protein of (7) above in each yeast; and selecting a test yeast having a large or small amount of the protein from then.

(23) A method for producing an alcoholic beverage comprising: conducting fermentation for producing an alcoholic beverage using the yeast according to any one of (10) to (13) or a yeast selected by the method according to any one of (20) to (22); and adjusting the production amount of ester.

### BRIEF DESCRIPTION OF DRAWINGS

FIG. 1 shows the cell growth with time, upon beer fermentation test. The horizontal axis represents fermentation time while the vertical axis represents optical density at 660 nm (OD660).

FIG. 2 shows the extract (sugar) consumption with time upon beer fermentation test. The horizontal axis represents fermentation time while the vertical axis represents apparent extract concentration (w/w %).

FIG. 3 shows the expression profile of nonScIAH1 gene in yeasts upon beer fermentation test. The horizontal axis represents fermentation time while the vertical axis represents the intensity of detected signal.

FIG. 4 shows the cell growth with time upon fermentation test The horizontal axis represents fermentation time while the vertical axis represents optical density at 660 nm (OD660). The symbol “IAH1” denotes a nonScIAH1 highly expressed strain.

FIG. 5 shows the extract (sugar) consumption with time upon beer fermentation test. The horizontal axis represents fermentation time while the vertical axis represents apparent extract concentration (w/w %). The symbol “IAH1” denotes a nonScIAH1 highly expressed sty

FIG. 6 shows the cell growth with time upon fermentation test. The horizontal axis represents fermentation time while the vertical axis represents optical density at 660 nm (OD660). The symbol “iah1” denotes a nonScIAH1 disrupted strain.

FIG. 7 shows the extract (sugar) consumption with time upon beer fermentation test The horizontal axis represents fermentation time while the vertical axis represents apparent extract concentration (w/w %). The symbol “iah1” denotes a nonScIAH1 disrupted strain

### BEST MODES FOR CARRYING OUT THE INVENTION

The present inventors conceived that it is possible to control ester in products by increasing or decreasing a esterase activity of the yeast. The present inventors have studied based on this conception and as a result, isolated and identified a nonScIAH1 gene encoding a esterase unique to lager brewing yeast based on the lager brewing yeast genome information mapped according to the method disclosed in Japanese Patent Application Laid-Open No. 2004-283169: The nucleotide sequence of the gene is represented by SEQ ID NO: 1. Further, an amino acid sequence of a protein encoded by the gene is represented by SEQ ID NO: 2.

### 1. Polynucleotide of the Invention

First of all, the present invention provides (a) a polynucleotide comprising a polynucleotide of the nucleotide sequence of SEQ ID NO: 1; and (b) a polynucleotide comprising a polynucleotide encoding a protein of the amino acid sequence of SEQ ID NO:2. The polynucleotide can be DNA or RNA.

The target polynucleotide of the present invention is not limited to the polynucleotide encoding a esterase gene derived from lager brewing yeast described above and may include other polynucleotides encoding proteins having equivalent functions to said protein. Proteins with equivalent functions include, for example, (c) a protein of an amino acid sequence of SEQ ID NO: 2 with one or more amino acids thereof being deleted, substituted, inserted and/or added and having a esterase activity.

Such proteins include a protein consisting of an amino acid sequence of SEQ ID NO: 2 with, for example, 1 to 100, 1 to 90, 1 to 80, 1 to 70, 1 to 60, 1 to 50, 1 to 40, 1 to 39, 1 to 38, 1 to 37, 1 to 36, 1 to 35, 1 to 34, 1 to 33, 1 to 32, 1 to 31, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6 (1 to several amino acids), 1 to 5, 1 to 4, 1 to 3, 1 to 2, or 1 amino acid residues thereof being deleted, substituted, inserted and/or added and having a esterase activity. In general the number of deletions, substitutions, insertions, and/or additions is preferably smaller. In addition, such proteins include (d) a protein having an amino acid sequence with about 60% or higher, about 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8%- or higher, or 99.90% or higher identity with the amino acid sequence of SEQ ID NO: 2, and having a esterase activity. In general the percentage identity is preferably higher.

Esterase activity may be measured, for example, by a method described in Appl. Microbiol. Biotechnol. 53: 596-600, 2000.

Furthermore, the present invention also contemplates (e) a polynucleotide comprising a polynucleotide which hybridizes to a polynucleotide consisting of a nucleotide sequence complementary to the nucleotide sequence of SEQ ID NO: 1 under stringent conditions and which encodes a protein having a esterase activity; and (f) a polynucleotide comprising a polynucleotide which hybridizes to a polynucleotide complementary to a nucleotide sequence of encoding a protein of SEQ ID NO: 2 under stringent conditions, and which encodes a protein having a esterase activity.

Herein, “a polynucleotide that hybridizes under stringent conditions” refers, to nucleotide sequence, such as a DNA, obtained by a colony hybridization technique, a plaque hybridization technique, a southern hybridization technique or the like using all or part of polynucleotide of a nucleotide sequence-complementary to the nucleotide sequence of SEQ ID NO: 1 or polynucleotide encoding the amino acid sequence of SEQ ID NO: 2 as a probe. The hybridization method may be a method described, for example, in MOLECULAR CLONING 3rd Ed., CURRENT PROTOCOLS IN MOLECULAR BIOLOGY, John Wiley & Sons 1987-1997, and so on.

The term “stringent conditions” as used herein may be any of low stringency conditions, moderate stringency conditions or high stringency conditions. “Low stringency conditions” are, for example, 5×SSC, 5×Denhardt's solution, 0.5% SDS, 500% formamide at 32° C. “Moderate stringency conditions” are, for example, 5×SSC, 5×Denhardt's solution, 0.5% SDS, 50% formamide at 42° C. “High stringency conditions” are, for example, 5×SSC, 5×Denhardt's solution, 0.5% SDS, 50% formamide at 50° C. Under these conditions, a polynucleotide, such as a DNA, with higher homology is expected to be obtained efficiently at higher temperature, although multiple factors are involved in hybridization stringency including temperature, probe concentration, probe length, ionic strength time, salt concentration and others, and one skilled in the art may appropriately select these factors to realize similar stringency.

When a commercially available kit is used for hybridization, for example, Alkphos Direct Labeling Reagents (Amersham Pharmacia) may be used. In this case, according to the attached protocol after incubation with a labeled probe overnight, the membrane is washed with a primary wash buffer coining 0.1% (w/v) SDS at 55° C., thereby detecting hybridized polynucleotide, such as DNA.

Other polynucleotides that can be hybridized include polynucleotides having about 60% or higher, about 70% or higher, 71% or higher, 72% or higher, 73% or higher, 74% or higher, 75% or higher, 76% or higher, 77% or higher, 78% or higher, 79% or higher, 0.80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher or 99.9% or higher identity to polynucleotide encoding the amino acid sequence of SEQ ID NO: 2 as calculated by homology search software, such as FASTA and BLAST using default parameters.

Identity between amino acid sequences or nucleotide sequences may be determined using algorithm BLAST by Karlin and Altschul (Proc. Natl. Acad. Sci. USA, 87: 2264-2268, 1990; Proc. Natl. Acad. Sci. USA, 90: 5873, 1993). Programs called BLASTN and BLASTX based on BLAST algorithm have been developed (Altschul S F et al, J. Mol. Biol. 215: 403, 1990). When a nucleotide sequence is sequenced using BLASTN, the parameters are, for example, score=100 and word length=12. When an amino acid sequence is sequenced using BLASTX the parameters are, for example, score=50 and word length=3. When BLAST and Gapped BLAST programs are used, default parameters for each of the programs are employed.

The polynucleotide of the present invention includes (j) a polynucleotide encoding RNA having a nucleotide sequence complementary to a transcript of the polynucleotide (DNA) according to (5) above; (k) a polynucleotide encoding RNA that represses the expression of the polynucleotide (DNA) according to (5) above through RNAi effect; (l) a polynucleotide encoding RNA having an activity of specifically cleaving a transcript of the polynucleotide (DNA) according to (5) above; and (m) a polynucleotide encoding RNA that represses expression of the polynucleotide (DNA) according to (5) above through co-suppression effect. These polynucleotides may be incorporated into a vector, which can be introduced into a cell for transformation to repress the expression of the polynucleotides (DNA) of (a) to (i) above. Thus, these polynucleotides may suitably be used when repression of the expression of the above DNA is preferable.

The phrase “polynucleotide encoding RNA having a nucleotide sequence complementary to the transcript of DNA” as used herein refers to so-called antisense DNA. Antisense technique is known as a method for repressing expression of a particular endogenous gene, and is described in various publications (see e.g., Hirajima and Inoue: New Biochemistry Experiment Course 2 Nucleic Acids IV Gene Replication and Expression (Japanese Biochemical Society Ed., Tokyo Kagaku Dozin Co., Ltd.) pp. 319-347; 1993). The sequence of antisense DNA is preferably complementary to all or part of the endogenous gene, but may not be completely complementary as long as it can effectively repress the expression of the gene. The transcribed RNA has preferably 90% or higher, and more preferably 95% or higher complementarity to the transcript of the target gene. The length of the antisense DNA is at least 15 bases or more, preferably 100 bases or more, and more preferably 500 bases or more.

The phrase “polynucleotide encoding RNA that represses DNA expression through RNAi effect” as used herein refers to a polynucleotide for repressing expression of an endogenous gene through RNA interference (RNAi). The term “RNAi” refers to a phenomenon where when double-stranded RNA having a sequence identical or similar to the target gene sequence is introduced into a cell, the expressions of both the introduced foreign gene and the target endogenous gene are repressed. RNA as used herein includes, for example, double-stranded RNA that causes RNA interference of 21 to 25 base length, for example, dsRNA (double strand RNA), siRNA (small interfering RNA) or shRNA (short hairpin RNA). Such RNA may be locally delivered to a desired site with a delivery system such as liposome, or a vector that generates the double-stranded RNA described above may be used for local expression thereof. Methods for producing or using such double-stranded RNA (dsRNA, siRNA or shRNA) are known from many publications (see, e.g., Japanese National Phase PCT Laid-open Patent Publication No. 2002-516062; US 2002/086356A; Nature Genetics, 24(2), 180-183, 2000 February; Genesis, 26(4), 240-244, 2000 April; Nature, 407:6802, 319-20, 2002 Sep. 21; Genes. & Dev., Vol. 16, (8), 948-958, 2002 Apr. 15; Proc. Natl. Acad. Sci. USA., 99(8), 5515-5520, 2002 Apr. 16; Science, 296(5567), 550-553, 2002 Apr. 19; Proc Natl. Acad. Sci. USA, 99:9, 6047-6052, 2002 Apr. 30; Nature Biotechnology, Vol. 20 (5), 497-500, 2002 May, Nature Biotechnology, Vol. 20(5), 500-505, 2002 May, Nucleic Acids Res., 30:10, e46, 2002 May 15).

The phase “polynucleotide encoding RNA having an activity of specifically cleaving transcript of DNA” as used herein generally refers to a ribozyme. Ribozyme is an RNA molecule with a catalytic activity that cleaves a transcript of a target DNA and inhibits the function of that gene. Design of ribozymes can be found in various known publications (see, e.g., FEBS Lett. 228: 228, 1988; FEBS Lett. 239: 285, 1988; Nucl. Acids. Res. 17: 7059, 1989; Nature 323: 349, 1986; Nucl. Acids. Res. 19: 6751, 1991; Protein Eng 3: 733, 1990; Nucl. Acids Res. 19: 3875, 1991; Nucl. Acids Res. 19: 5125, 1991; Biochem Biophys Res Commun 186: 1271, 1992). In addition, the phrase “polynucleotide encoding RNA that represses DNA expression through co-suppression effect” refers to a nucleotide that inhibits functions of target DNA by “co-suppression”.

The term “co-suppression” as used herein, refers to a phenomenon where when a gene having a sequence identical or similar to a target endogenous gene is transformed into a cell the expressions of both the introduced foreign gene and the target endogenous gene are repressed. Design of polynucleotides having a co-suppression effect can also be found in various publications (see, e.g., Smyth DR: Curr. Biol. 7: R793, 1997, Martienssen R: Curr. Biol. 6: 810, 1996).

2. Protein of the Present invention

The present invention also provides proteins encoded by any of the polynucleotides (a) to (i) above. A preferred protein of the present invention comprises an amino acid sequence of SEQ ID NO:2 with one or several amino acids thereof being deleted, substituted, inserted and/or added, and has a esterase activity.

Such protein includes those having an amino acid sequence of SEQ ID NO: 2 with amino acid residues thereof of the number mentioned above being deleted, substituted, inserted and/or added and having a esterase activity. In addition, such protein includes those having homology as described above with the amino acid sequence of SEQ ID NO: 2 and having a esterase activity.

Such proteins may be obtained by employing site directed mutation described, for example, in MOLECULAR CLONING 3rd Ed., CURRENT PROTOCOLS IN MOLECULAR BIOLOGY, Nuc. Acids. Res., 10: 6487 (1982), Proc. Natl. Acad. Sci. USA 79: 6409 (1982), Gene 34: 315 (1985), Nuc. Acids. Res., 13: 4431 (1985), Proc. Natl. Acad. Sci. USA 82: 488 (1985).

Deletion, substitution, insertion and/or addition of one or more amino acid residues in an amino acid sequence of the protein of the invention means that one or more amino acid residues are deleted, substituted, inserted and/or added at any one or more positions in the same amino acid sequence. Two or more types of deletion, substitution, insertion and/or addition may occur concurrently.

Hereinafter, examples of mutually substitutable amino acid residues are enumerated. Amino acid residues in the same group are mutually substitutable. The groups are provided below.

Group A: leucine, isoleucine, norleucine, valine, norvaline, alanine, 2-aminobutanoic acid, methionine, o-methylserine, t-butylglycine, t-butylalanine, cyclohexylalanine; Group B: asparatic acid, glutamic acid, isoasparatic acid, isoglutamic acid, 2-aminoadipic acid, 2-aminosuberic acid; Group C: asparagine, glutamine; Group D: lysine, arginine, ornithine, 2,4-diaminobutanoic acid, 2,3-diaminopropionic acid; Group E: proline, 3-hydroxyproline, 4-hydroxyproline; Group F: serine, threonine, homoserine; and Group G: phenylalanine, tyrosine.

The protein of the present invention may also be produced by chemical synthesis methods such as Fmoc method (fluorenylmethyloxycarbonyl method) and tBoc method (t-butyloxycarbonyl method). In addition, peptide synthesizers available from, for example, Advanced ChemTech, PerkinElmer, Phamiacia, Protein Technology Instrument, Synthecell-Vega, PerSeptive, Shimazu Corp. can also be used for chemical synthesis.

3. Vector of the Invention and Yeast Transformed with the Vector

The present invention then provides a vector comprising the polynucleotide described above. The vector of the present invention is directed to a vector including any of the polynucleotides described in (a) to (i) above or the polynucleotides described in (j) to (m) above. Generally, the vector of the present invention comprises an expression cassette including as components (x) a promoter that can transcribe in a yeast cell; (y) a polynucleotide described in any of (a) to (i) above that is linked to the promoter in sense or antisense direction; and (z) a signal that functions in the yeast with respect to transcription termination and polyadenylation of RNA molecule.

According to the present invention, in order to highly express the protein of the invention described above upon brewing alcoholic, beverages (e.g., beer) described below, these polynucleotides are introduced in the sense direction to the promoter to promote expression of the polynucleotide (DNA) described in any of (a) to (i) above. Further, in order to repress the above protein of the invention upon brewing alcoholic beverages (e.g., beer) described below, these polynucleotides are introduced in the antisense direction to the promoter to repress the expression of the polynucleotide (DNA) described in any of (a) to (i) above. In order to repress the above protein of the invention, the polynucleotide may be introduced into vectors such that the polynucleotide of any of the (j) to (m) is to be expressed. According to the present invention, the target gene (DNA) may be disrupted to repress the expression of the DNA described above or the expression of the protein described above. A gene may be disrupted by adding or deleting one or more bases to or from a region involved in expression of the gene product in the target gene, for example, a coding region or a promoter region, or by deleting these regions entirely. Such disruption of gene may be found in known publications (see, e.g., Proc. Natl. Acad. Sci USA, 76, 4951 (1979), Methods in Enzymology, 101, 202 (1983), Japanese Patent Application Laid-Open No. 6-253826).

A vector introduced in the yeast may be any of a multicopy type (YEp type), a single copy type (YCp type), or a chromosome integration type (YIp type). For example, YEp24 (J. R Broach et al, EXPERIMENTAL MANIPULATION OF GENE EXPRESSION, Academic Press, New York, 83, 1983) is known as a YEp type vector, YCp50 (ML D. Rose et al., Gene 60: 237, 1987) is known as a YCp type vector, and YIp5 (K. Struhl et al., Proc. Natl. Acad. Sci. USA, 76: 1035, 1979) is known as a YIp type vector, all of which are readily available.

Promoters/terminators for adjusting gene expression in yeast may be in any combination as long as they function in the brewery yeast and they are not influenced by constituents in fermentation broth. For example, a promoter of glyceraldehydes 3-phosphate dehydrogenase gene (TDH3), or a promoter of 3-phosphoglycerate kinase gene (PGK1) may be used. These genes have previously been cloned, described in detail for example, in F. Tuite et al., EMBO J., 1, 603 (1982), and are readily available by known methods.

Since an auxotrophy marker cannot be used as a selective marker upon transformation for a brewery yeast, for example, a geneticin-resistant gene (G418r), a copper-resistant gene (CUP1) (Marin et al., Proc. Natl. Acad. Sci. USA, 81, 337 1984) or a cerulenin-resistant gene (fas2m, PDR4) (Junji Inokoshi et al., Biochemistry, 64, 660, 1992; and Hussain et al., Gene, 101: 149, 1991, respectively) may be used.

A vector constructed as described above is introduced into a host yeast. Examples of the host yeast include any yeast that can be used for brewing, for example, brewery yeasts for beer, wine and sake. Specifically, yea such as genus Saccharomyces may be used. According to the present invention, a lager brewing yeast, for example, Saccharomyces pastorianus W34/70, etc., Saccharomyces carlsbergensis NCYC453 or NCYC456, etc., or Saccharomyces cerevisiae NBRC1951, NBRC1952, NBRC1953 or NBRC1954, etc., may be used. In addition, whisky yeasts such as Saccharomyces cerevisiae NCYC90, wine yeasts such as wine yeast #1, 3 and 4 from the Brewing Society of Japan, and sake yeasts such as sake yeast #7 and 9 from the Brewing Society of Japan may also be used but not limited thereto. In the present invention, lager brewing yeasts such as Saccharomyces pastorianus may be used preferably.

A yeast transformation method may be a generally used known method. For example, methods that can be used include but not limited to an electroporation method (Meth. Enzym., 194: 182 (1990)), a spheroplast method (Proc. Natl. Acad. Sci. USA, 75: 1929 (1978)), a lithium acetate method (J. Bacteriology, 153: 163 (1983)), and methods described in Proc. Natl. Acad. Sci. USA, 75: 1929 (1978), METHODS IN YEAST GENETICS, 2000 Edition: A Cold Spring Harbor Laboratory Course Manual.

More specifically, a host yeast is cultured in a standard yeast nutrition medium (e.g., YEPD medium (Genetic Engineering. Vol. 1, Plenum Press, New York 117 (1979)), etc.) such that OD600 nm will be 1 to 6. This culture yeast is collected by centrifugation, washed and pretreated with alkali metal ion, preferably lithium ion at a concentration of about 1 to 2 M. After the cell is left to stand at about 30° C. for about 60 minutes, it is left to stand with DNA to be introduced (about 1 to 20 μg) at about 30° C. for about another 60 minutes. Polyethyleneglycol, preferably about 4,000 Dalton of polyethyleneglycol, is added to a final concentration of about 20% to 50%. After leaving at about 30° C. for about 30 minutes, the cell is heated at about 42° C. for about 5 minutes. Preferably, this cell suspension is washed with a standard yeast nutrition medium, added to a predetermined amount of fresh standard yeast nutrition medium and left to stand at about 30° C. for about 60 minutes. Thereafter, it is seeded to a standard agar medium containing an antibiotic or the like as a selective marker to obtain a transformant.

Other general cloning techniques may be found, for example, in MOLECULAR CLONING 3rd Ed, and METHODS IN YEAST GENETICS, A LABORATORY MANUAL (Cold Spring Harbor Laboratory Press, Cold Spring Harbor, N.Y.).

### 4. Method of Producing Alcoholic Beverages According to the Present Invention and Alcoholic Beverages Produced by the Method

The vector of the present invention described above is introduced into a yeast suitable for brewing a target alcoholic product. This yeast can be used to produce a desired alcoholic beverage with enhanced aroma and flavor with an elevated content of ester. In addition, yeasts to be selected by the yeast assessment method of the present invention described below can also be used. The target alcoholic beverages include, for example, but not limited to beer, beer-taste beverages such as sparkling liquor (happoushu), wine, whisky, sake and the like. Further, according to the present invention, desired alcoholic beverages with reduced ester level can be produced using brewery yeast in which the expression of the target gene was suppressed, if needed. That is to say, desired kind of alcoholic beverages with controlled (elevated or reduced) level of ester can be produced by controlling (elevating or reducing) production amount of ester using yeasts into which the vector of the present invention was introduced described above, yeasts in which expression of the polynucleotide (DNA) of the present invention described above was suppressed or yeasts selected by the yeast assessment method of the invention described below for fermentation to produce alcoholic beverages.

In order to produce these alcoholic beverages, a known technique can be used except that a brewery yeast obtained according to the present invention is used in the place of a parent strain. Since materials, manufacturing equipment, manufacturing control and the like may be exactly the same as the conventional ones, there is no need of increasing the cost for producing alcoholic beverages with an controlled content of ester. Thus, according to the present invention, alcoholic beverages with excellent aroma and flavor can be produced using the existing facility without increasing the cost.

### 5. Yeast Assessment Method of the Invention

The present invention relates to a method for assessing a test yeast for its ester-producing capability by using a primer or a probe designed based on a nucleotide sequence of a esterase gene having the nucleotide sequence of SEQ ID NO:1. General techniques for such assessment method is known and is described in, for example, WO01/040514, Japanese Laid-Open Patent Application No. 8-205900 or the like. This assessment method is described in below.

First, genome of a test yeast is prepared. For this preparation, any known method such as Hereford method or potassium acetate method may be used (e.g., METHODS IN YEAST GENETICS, Cold Spring Harbor Laboratory Press, 130 (1990)). Using a primer or a probe designed based on a nucleotide sequence (preferably, ORF sequence) of the esterase gene, the existence of the gene or a sequence specific to the gene is determined in the test yeast genome obtained. The primer or the probe may be designed according to a known technique.

Detection of the gene or the specific sequence may be carried out by employing a known technique. For example, a polynucleotide including part or all of the specific sequence or a polynucleotide including a nucleotide sequence complementary to said nucleotide sequence is used as one primer, while a polynucleotide including part or all of the sequence upstream or downstream from this sequence or a polynucleotide including a nucleotide sequence complementary to said nucleotide sequence, is used as another primer to amplify a nucleic acid of the yeast by a PCR method, thereby determining the existence of amplified products and molecular weight of the amplified products. The number of bases of polynucleotide used for a primer is generally 10 base pairs (bp) or more, and preferably 15 to 25 bp. In general, the number of bases between the primers is suitably 300 to 2000 bp.

The reaction conditions for PCR are not particularly limited but may be, for example a denaturation temperature of 90 to 95° C., an annealing temperature of 40 to 60° C., an elongation temperature of 60 to 75° C., and the number of cycle of 10 or more. The resulting reaction product may be separated, for example, by electrophoresis using agarose gel to determine the molecular weight of the amplified product. This method allows prediction and assessment of the capability of the yeast to produce ester as determined by whether the molecular weight of the amplified product is a size that contains the DNA molecule of the specific part. In addition, by analyzing the nucleotide sequence of the amplified product, the capability may be predicted and/or assessed more precisely.

Moreover, in the present invention, a test yeast is cultured to measure an expression level of the esterase gene having the nucleotide sequence of SEQ ID NO: 1 to assess the test yeast for its ester-producing capability. In measuring an expression level of the esterase gene, the test yeast is cultured and then mRNA or a protein resulting from the esterase gene is quantified. The quantification of mRNA or protein may be carried out by employing a known technique. For example, mRNA may be quantified, by Northern hybridization or quantitative RT-PCR, while protein may be quantified, for example, by Western blotting (CURRENT PROTOCOLS IN MOLECULAR BIOLOGY, John Wiley & Sons 19942003).

Furthermore, test yeasts are cultured and expression levels of the esterase gene having the nucleotide sequence of SEQ ID NO: 1 are measured to select a test yeast with the gene expression level according to the target capability of producing ester, thereby selecting a yeast favorable for brewing desired alcoholic beverages. In addition, a reference yeast and a test yeast may be cultured so as to measure and compare the expression level of the gene in each of the yeasts, thereby selecting a favorable test yeast. More specifically, for example, a reference yeast and one or more test yeasts are cultured and an expression level of the esterase gene having the nucleotide sequence of SEQ ID NO: 1 is measured in each yeast. By selecting a test yeast with the gene expressed higher or lower than that in the reference yeast, a yeast suitable for brewing alcoholic beverages can be selected.

Alternatively, test yeasts are cultured and a yeast with a higher or lower ester-producing capability or with a higher or lower esterase activity is selected, thereby selecting a yeast suitable for brewing desired alcoholic beverages.

In these cases, the test yeasts or the reference yeast may be, for example, a yeast introduced with the vector of the invention, a yeast in which an expression of a polynucleotide (DNA) of the invention has been controlled, an artificially mutated yeast or a naturally mutated yeast. The ester-producing capability can be measured by, for example, a method described in Method of J. Am. Soc. Brew. Chem. 49:152-157, 1991. Esterase activity can be measured by, for example, a method described in Appl. Microbiol. Biotechnol. 53: 596-600, 2000. The mutation treatment may employ any methods including, for example, physical methods such as ultraviolet irradiation and radiation irradiation, and chemical methods associated with treatments with drugs such as EMS (ethylmethane sulphonate) and N-methyl-N-nitrosoguanidine (see, e-g, Yasuji Oshima Ed., BIOCHEMISTRY EXPERIMENTS is vol. 39, Yeast Molecular Genetic Experiments, pp. 67-75, JSSP).

In addition, examples of yeasts used as the reference yeast or the test yeasts include any yeasts that can be used for brewing, for examples brewery yeasts for beer, wine, sake and the like. More specifically, yeasts such as genus Saccharomyces may be used (e.g., S. pastorianus, S. cerevisiae, and S. carlsbergensis). According to the present invention, a lager brewing yeast, for example, Saccharomyces pastorianus W34170; Saccharomyces carlsbergensis NCYC453 or NCYC456; or Saccharomyces cerevisiae NBRC1951, NBRC1952, NBRC1953 or NBRC1954, etc., may be used. Further, wine yeasts such as wine yeasts #1,3 and 4 from the Brewing Society of Japan; and sake yeasts such as sake yeast #7 and 9 from the Brewing Society of Japan may also be used but not limited thereto. In the present invention, lager brewing yeasts such as Saccharomyces pastorianus may preferably be used. The reference yeast and the test yeasts may be selected from the above yeasts in any combination

### EXAMPLES

Hereinafter, the present invention will be described in more detail with reference to working examples. The present invention, however, is not limited to the examples described below.

### Example 1

### Cloning of Esterase Gene (nonScIAH1)

A specific novel esterase gene (nonScIAH1) (SEQ ID NO: 1) from a lager brewing yeast were found, as a result of a search utilizing the comparison database described in Japanese Patent Application Laid-Open No. 2004-283169. Based on the acquired nucleotide sequence information, primers nonScIAH1_for (SEQ ID NO: 3) and nonScIAH1_rv (SEQ ID NO: 4) were designed to amplify the full-length genes, respectively. PCR was carried out using chromosomal DNA of a genome sequencing strain, Saccharomyces pastorianus Weihenstephan 34/70 strain, also abbreviated to “W34/70 strain”, as a template to obtain DNA fragments (about 0.7 kb) including the full-length gene of nonScIAH1.

The thus-obtained nonScIAH1 gene fragment was inserted into pCR2.1-TOPO vector (Invitrogen) by TA cloning. The nucleotide sequences of nonScIAH1 gene were analyzed according to Sanger's method (F. Sanger, Science, 214: 1215, 1981) to confirm the nucleotide sequence.

### Example 2

### Analysis of Expression of nonScIAH1 Gene during Beer Fermentation Test

A beer fermentation test was conducted using a lager brewing yeast, Saccharomyces pastorianus W34/70 strain and then mRNA extracted from yeast cells during fermentation was analyzed by a DNA microarray.

| Wort extract concentration | 12.69% | |
|---|---|---|
| Wort content | 70 | L |
| Wort dissolved oxygen concentration | 8.6 | ppm |
| Fermentation temperature | 15° | C. |
| Yeast pitching rate | 12.8 × 10⁶ | cells/mL |

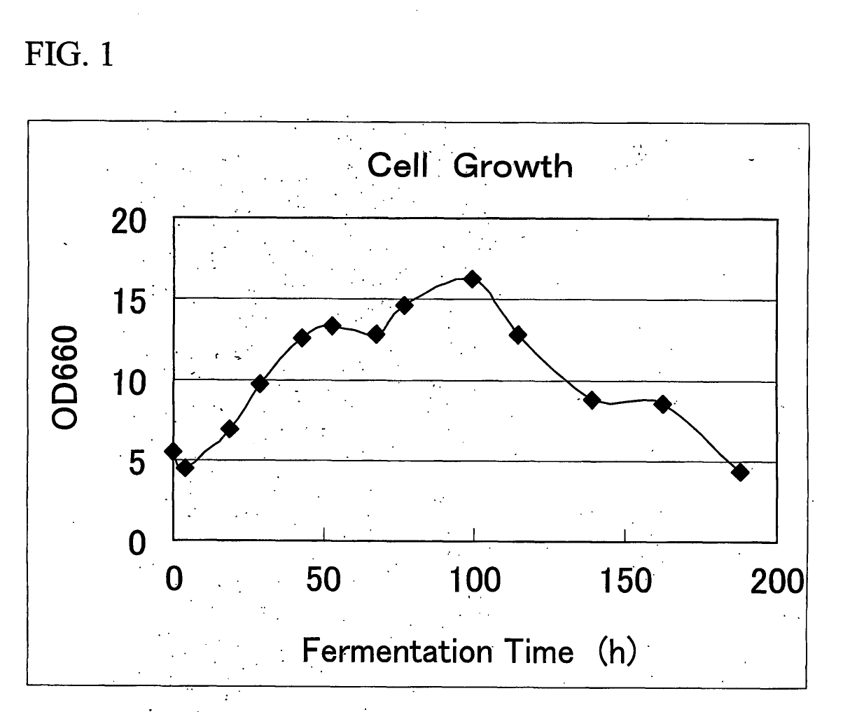

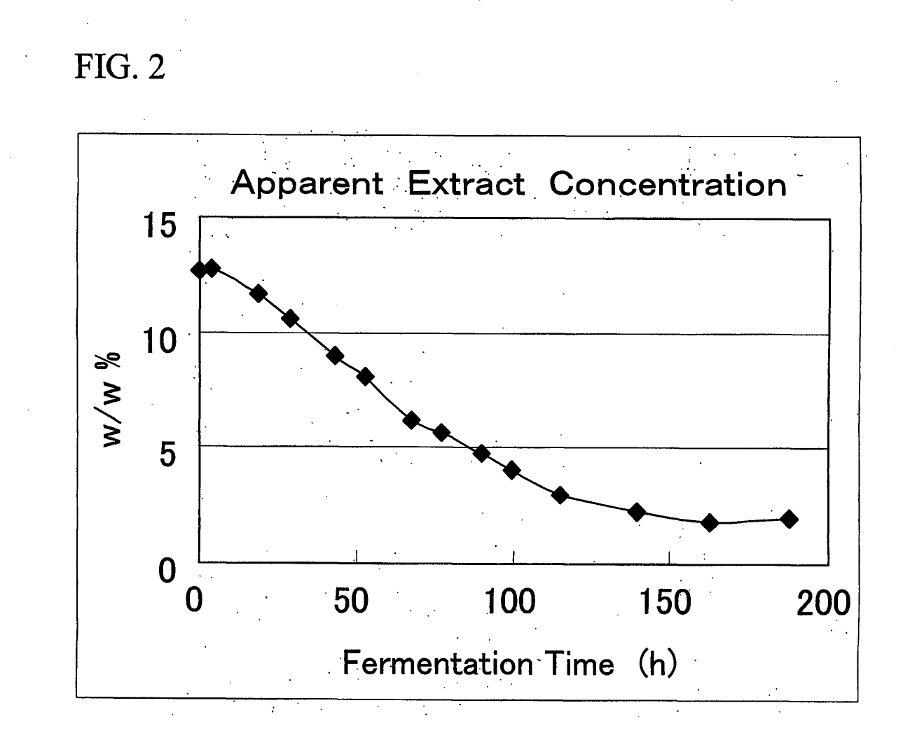

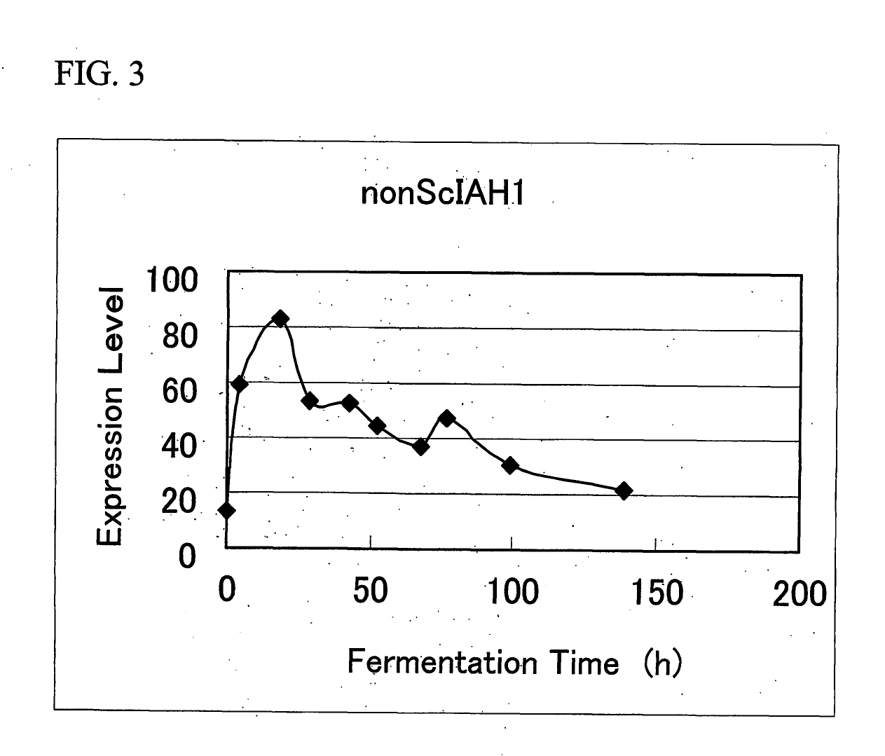

Sampling of fermentation liquor was performed with time, and variation with time of yeast growth amount (FIG. 1) and apparent extract concentration (FIG. 2) was observed. Simultaneously, sampling of yeast cells was performed, and the prepared mRNA was subjected to be biotin-labeled and was hybridized to a beer yeast DNA microarray. The signal was detected using GCOS; GeneChip Operating Software 1.0 (manufactured by Affymetrix Co.). Expression pattern of nonScIAH1 gene is shown in FIG. 3. As a result, it was confirmed that nonScIAH1 gene was expressed in the general beer fermentation.

### Example 3

### Construction of nonScIAH1 Gene Highly Expressed Strain

The nonScIAH1/pCR2.1-TOPO described in Example 1 was digested using the restriction enzymes SacI and NotI so as to prepare a DNA fragment containing the entire length of the protein-encoding region. This fragment was ligated to pYCGPYNot treated with the restriction enzymes SacI and NotI, thereby constructing the nonScIAH1 high expression vector nonScIAH1/pYCGPYNot. pYCGPYNot is the YCp-type yeast expression vector. The inserted gene is highly expressed by the pyruvate kinase gene PYK1 promoter. The geneticin-resistant gene G418^{r} is included as the selection marker in the yeast, and the ampicillin-resistant gene Amp^{r} is included as the selection marker in Escherichia coli.

Using the high expression vector prepared by the above method, the stain Saccharomyces pasteurianus Weihenstephaner 34/70 was transformed by the method described in Japanese Patent Application Laid-open No. H7-303475. The transformant was selected in a YPD plate culture (1% yeast extract, 2% polypeptone, 2% glucose, 2% agar) containing 300 mg/L of geneticin.

### Example 4

### Analysis of Amounts of Ester Formed in Beer Fermentation Test

A fermentation test was conducted under the following conditions using the parent strain (W34/70 strain) and the nonScIAH1 highly expressed strain obtained in Example 3.

Wort extrct concentration: 12%

Wort volume: 2 L

Wort dissolved oxygen concentration: 8 ppm

Fermentation temperature: 15° C.

Yeast pitching rate: 5 g/L

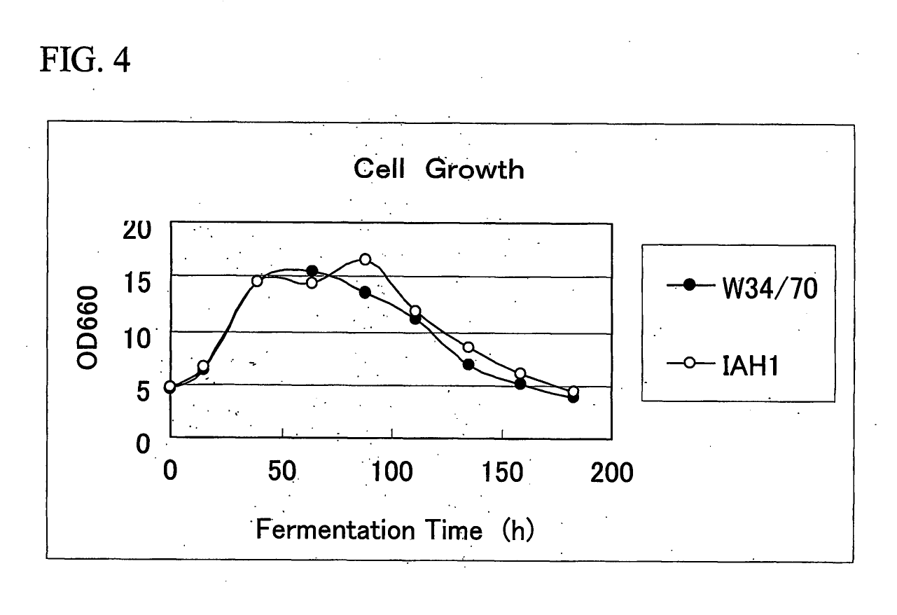

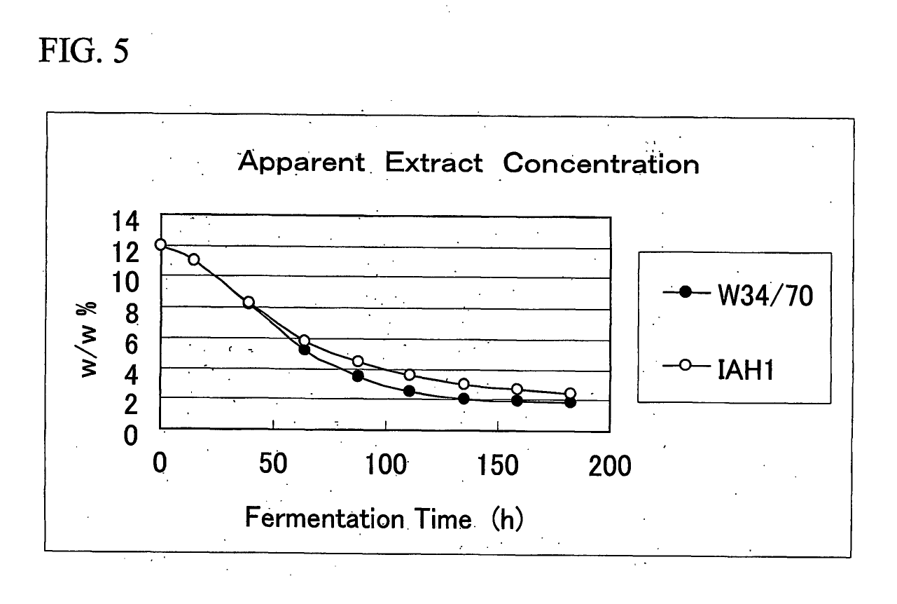

The fermentation broth was sampled over tine to investigate the time-based changes in yeast growth (OD660) (FIG. 4) and extract consumption (FIG. 5). Quantification of higher alcohol and extract concentrations at completion of fermentation was carried out using head space gas chromatography (J. Am. Soc. Brew. Chem. 49:152-157 1991).

The amount of ethyl acetate formed at completion of fermentation was 24.0 ppm for the nonScIAH1 highly expressed strain in contrast to 34.4 ppm for the parent strain as described in Table 1. The amount of isoamyl acetate formed was 0.2 ppm for the nonScIAH1 highly expressed strain in contrast to 2.1 ppm for the parent strain. On the basis of these results, the amounts of ethyl acetate and isoamyl acetate formed were clearly demonstrated to decreased by 30 to 90% by high expression of nonScIAH1.

TABLE 1

| | Parent Strain | | |
|---|---|---|---|
| | (W34/70) | nonScIAH1 Highly Expressed Strain | |
| Ethyl acetate | 34.4 | 24.0 | (70%) |
| Isoamyl acetate | 2.1 | 0.2 | (9.5%) |

Unit: ppm
Values in parentheses indicate relative values versus the parent strain.

### Example 5

### Disruption of nonScIAH1 Gene

Fragments for gene disruption were prepared by PCR using plasmids containing a drug resistance marker, (pFA6a(G418r), pAG25(nat1), pAG32(hph)) as templates in accordance with a method described in the literature (Goldstein et al., Yeast, 15,1541 (1999)). Primers consisting of nonScIAH1_delta_for (SEQ ID NO. 5N), and nonScIAH1_delta_rv (SEQ ID NO. 6) were used for the PCR primers.

A spore clone (W34/70-2) isolated from brewer's yeast Saccharomyces pastorianus strain W34/70 was transformed with the fragments for gene disruption prepared as described above. Transformation was carried out according to the method described in Japanese Patent Application Laid-open No. H07-303475, and transformants were selected on YPD plate medium (1% yeast extract, 2% polypeptone, 2% glucose, 2% agar) containing geneticin at 300 mg/L, nourseothricin at 50 mg/L or hygromycin B at 200 mg/L.

### Example 6

### Analysis of Amounts of Ester Formed in Beer Fermentation Test

A fermentation test was conducted under the following conditions using the parent strain (W34/70-2 strain) and the nonScIAH1 disrupted strain obtained in Example 5.

Wort extract concentration: 13%

Wort volume: 1 L

Wort dissolved oxygen concentration: 8 ppm

Fermentation temperature: 15° C.

Yeast pitching rate: 5 g/L

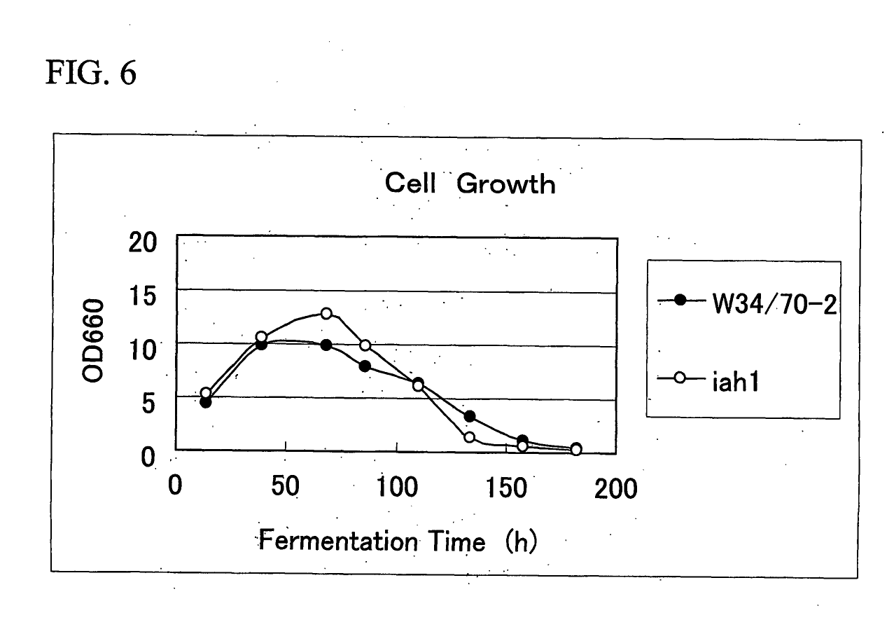

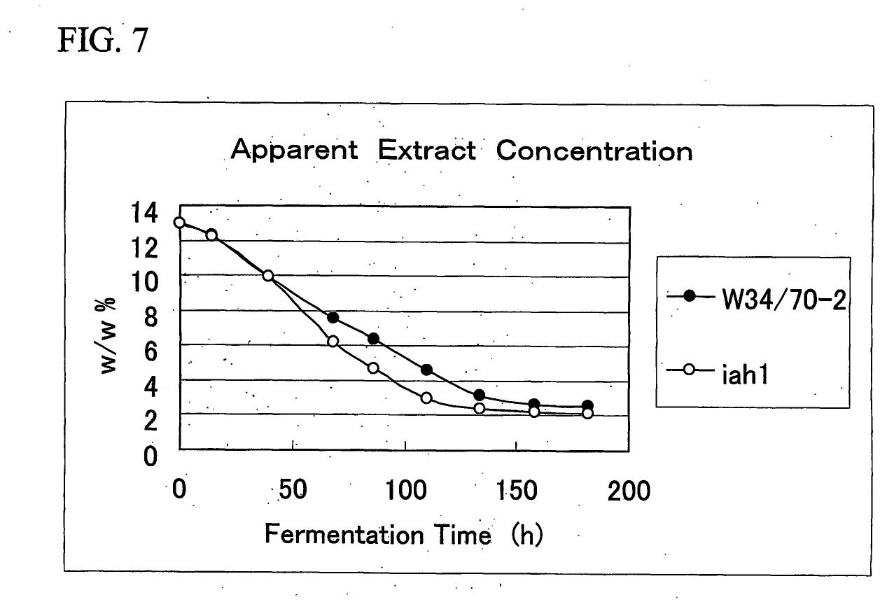

The fermentation broth was sampled over time to investigate the time-based changes in yeast growth (OD660) (FIG. 6) and extract consumption (FIG. 7). Quantification of ester concentration at completion of fermentation was carried out using head space gas chromatography (J. Am. Soc. Brew. Chem. 49:152-157, 1991).

The amount of ethyl acetate formed at completion of fermentation was 28.1 ppm for the nonScIAH1 disrupted strain in contrast to 26.2 ppm for the parent strain as described in Table 2. The amount of isoamyl acetate formed was 2.9 ppm for the nonScIAH1 disrupted strain in contrast to 2.3 ppm for the parent strain. On the basis of these results, the amounts of ethyl acetate and isoamyl acetate formed were clearly demonstrated to increased by 7 to 26% by disruption of nonScIAH1.

TABLE 2

| | Parent Strain | | |
|---|---|---|---|
| | (W34/70-2) | nonScIAH1 disrupted Strain | |
| Ethyl acetate | 26.2 | 28.1 | (107%) |
| Isoamyl acetate | 2.3 | 2.9 | (126%) |

Unit: ppm
Values in parentheses indicate relative values versus the parent strain.

### INDUSTRIAL APPLICABILITY

According to the alcoholic beverage production method of the present invention, alcoholic beverages having superior aroma and flavor can be produced because the method can control the content of esters.

More specifically, according to the alcoholic beverage production method of the present invention, alcoholic beverages having superior aroma and flavor can be produced because the method can increase the content of esters which impart a florid aroma to products. In addition, in the case of malt beverages such as beer, for which an excessively high ester content is not preferred, alcoholic beverages having a more desirable aroma and flavor can be produced because the method can also decrease the amount of ester contained therein.

1. A polynucleotide selected from the group consisting of:
(a) a polynucleotide comprising a polynucleotide consisting of the nucleotide sequence of SEQ ID NO: 1;
(b) a polynucleotide comprising a polynucleotide encoding a protein consisting of the amino acid sequence of SEQ ID NO:2;
(c) a polynucleotide comprising a polynucleotide encoding a protein consisting of the amino acid sequence of SEQ ID NO:2 with one or more amino acids thereof being deleted, substituted, inserted and/or added, and having a esterase activity;
(d) a polynucleotide comprising a polynucleotide encoding a protein having an amino acid sequence having 60% or higher identity with the amino acid sequence of SEQ ID NO:2, and having a esterase activity;
(e) a polynucleotide comprising a polynucleotide which hybridizes to a polynucleotide consisting of a nucleotide sequence complementary to the nucleotide sequence of SEQ ID NO: 1 under stringent conditions, and which encodes a protein having a esterase activity; and
(f) a polynucleotide comprising a polynucleotide which hybridizes to a polynucleotide consisting of a nucleotide sequence complementary to the nucleotide sequence of the polynucleotide encoding the protein of the amino acid sequence of SEQ ID NO:2 under stringent conditions, and which encodes a protein having a esterase activity.

2. The polynucleotide of claim 1 selected from the group consisting of:
(g) a polynucleotide encoding a protein consisting of the amino acid sequence of SEQ ID NO: 2, or encoding an amino acid sequence of SEQ ID NO: 2 wherein 1 to 10 amino acids thereof is deleted, substituted, inserted, and/or added, and wherein said protein has a esterase activity;
(h) a polynucleotide encoding a protein having 90% or higher identity with the amino acid sequence of SEQ ID NO: 2, and having a esterase activity; and
(i) a polynucleotide which hybridizes to SEQ ID NO: 1 or which hybridizes to a nucleotide sequence complementary to the nucleotide sequence of SEQ ID NO: 1 under high stringent conditions, and which encodes a protein having a esterase activity.

3. The polynucleotide of claim 1 comprising a polynucleotide consisting of SEQ ID NO: 1.

4. The polynucleotide of claim 1 comprising a polynucleotide encoding a protein consisting of SEQ ID NO: 2.

5. The polynucleotide of claim 1, wherein the polynucleotide is DNA.

6. A polynucleotide selected from the group consisting of:
(j) a polynucleotide encoding RNA of a nucleotide sequence complementary to a transcript of the polynucleotide (DNA) according to claim 5;
(k) a polynucleotide encoding RNA that represses the expression of the polynucleotide (DNA) according to claim 5 through RNAi effect;
(l) a polynucleotide encoding RNA having an activity of specifically cleaving a transcript of the polynucleotide (DNA) according to claim 5; and
(m) a polynucleotide encoding RNA that represses expression of the polynucleotide
(DNA) according to claim 5 through co-suppression effect.

7. A protein encoded by the polynucleotide of claim 1.

8. A vector comprising the polynucleotide of claim 1.

9. A vector comprising the polynucleotide of claim 6.

10. A yeast comprising the vector of claim 8.

11. The yeast of claim 10, wherein a ester-producing ability is decreased by introducing the vector comprising the polynucleotide.

12. A yeast, wherein an expression of the polynucleotide
(DNA) of claim 5 is repressed by introducing the vector comprising the polynucleotide, or by disrupting a gene related to the polynucleotide (DNA) of claim 5.

13. The yeast of claim 11, wherein a ester-producing ability is decreased by increasing an expression level of the protein encoded by the polynucleotide.

14. A method for producing an alcoholic beverage by using the yeast of claim 10.

15. The method for producing an alcoholic beverage of claim 14, wherein the brewed alcoholic beverage is a malt beverage.

16. The method for producing an alcoholic beverage of claim 14, wherein the brewed alcoholic beverage is wine.

17. An alcoholic beverage produced by the method of claim 14.

18. A method for assessing a test yeast for its ester-producing capability, comprising using a primer or a probe designed based on a nucleotide sequence of a esterase gene having the nucleotide sequence of SEQ ID NO: 1.

19. A method for assessing a test yeast for its ester-producing capability, comprising: culturing a test yeast; and measuring an expression level of a esterase gene having the nucleotide sequence of SEQ ID NO: 1.

20. A method for selecting a yeast, comprising: culturing test yeasts; quantifying the protein according to claim 7 or measuring an expression level of a esterase gene having the nucleotide sequence of SEQ ID NO: 1; and selecting a test yeast having said protein amount or said gene expression level according to a target capability of producing ester.

21. The method for selecting a yeast according to claim 20, comprising: culturing a reference yeast and test yeasts; measuring an expression level of a esterase gene having the nucleotide sequence of SEQ ID NO: 1 in each yeast; and selecting a test yeast having the gene expressed higher or lower than that in the reference yeast.

22. The method for selecting a yeast according to claim 20, comprising: culturing a reference yeast and test yeasts; quantifying the protein encoded by the polynucleotide in each yeast; and selecting a test yeast having said protein for a larger or smaller amount than that in the reference yeast.

23. A method for producing an alcoholic beverage comprising: conducting fermentation for producing an alcoholic beverage using the yeast according to claim 10 or a yeast selected by the method according to claim 20; and adjusting the production amount of ester.
